(19) United States
(12) Patent Application Publication
(10) Pub. No.: US 2008/0057584 A1
(43) Pub. Date: Mar. 6, 2008

(54) Intervention In The Energy Metabolism Of Eukaryote Cells For The Purposes Of Selection And Increased Expression

(76) Inventors: Volker Sandig, Berlin (DE); Ingo Jordan, Berlin (DE); Karsten Winkler, Berlin (DE)

Correspondence Address: NEEDLE & ROSENBERG, P.C., SUITE 1000, 999 PEACHTREE STREET, ATLANTA, GA, 30309-3915, US

(21) Appl. No.: 11/578,567

(22) PCT Filed: Apr. 15, 2005
(86) PCT No.: PCT/EP05/51679

§ 371 Date: Feb. 27, 2007

(30) Foreign Application Priority Data
Apr. 16, 2004 (EP) 04009094.6

(51) Int. Cl. C12N 15/87; C07H 21/04; C12N 5/06

(52) U.S. Cl. 435/455; 435/325; 536/23.1

(57) ABSTRACT

The invention relates to a method for expressing one or more foreign genes in eukaryote cells, wherein the energy metabolism of the cells is improved by introducing nucleic acid sequences which encode sugar carrier proteins and whose expression is linked to the expression of the foreign gene. The invention also relates to suitable transformed cells and to the use thereof. More particularly the nucleic acid sequences encode glucose carrier proteins such as GLUT-1.

The invention pertains to a process for the expression of one or more foreign genes in eukaryote cells, wherein the cell's energy metabolism is improved by the incorporation of nucleic acid sequences coding sugar carrier proteins and wherein the expression of said sequences is linked with the expression of the foreign genes, transformed cells suited therefore and the use thereof. Said nucleic acid sequences are coding in particular for glucose carrier proteins such as GLUT-1.

### BACKGROUND OF THE INVENTION

Important parameters for the production of recombinant active substances in cell cultures are (1) the stable maintenance of the foreign gene, (2) a high expression rate of the foreign gene and (3) the proliferation properties of the transformed cell. The invention described herein can contribute to the optimization of all three parameters.

Usually, foreign genes are expressed in eukaryote cells by a transfection of plasmids carrying the respective gene under the control of cis-active sequences such as the promoter and the polyadenylation signal. However, this process only results in a temporary or transient expression of foreign genes since the daughter cells are provided with increasingly less plasmids due to the division of transfected cells and by degradation of the inserted DNA. Hence, the transfected plasmids may not be detectable any more within a few passages. Thus, for a stable expression the foreign gene is required either to be integrated into the chromosome of the host cell—mediated by functions in the plasmid backbone—or its replication must be episomally linked to the cell division (Van Craenenbroeck, K. et al., Eur. J. Biochem. 267:5665-78 (2000)).

In most cases an expression will not be permanent also in the stable configuration: during cell division episomes may unevenly be distributed over daughter cells and hence be lost; chromosomal sections with the integrated foreign genes may be rendered inaccessible for the cellular transcription machinery by methylation and condensation or deleted by recombination. Since the introduction of foreign genes often results in a stress on the host cell, the cells wherein the inactivation of the foreign gene occurs are privileged over the stable transfectants. Hence, the revertants displace their transgenic neighbours during the passage of a cell line.

The occurrence of revertants may be counteracted by combining the expression of the foreign gene with a simultaneous expression of a selection marker.

Said selection marker may be a resistance factor being capable of protecting the host cell from antibiotics which are toxic for the wild type. Although this selection type produces very good results, some serious drawbacks have to be considered: (1) only partially inactivated antibiotics as well as the inactivation process as such stress the cell and may hence in theory lower possible expression rates to significantly lower levels. (2) Antibiotics reacted by the resistance factor have to be replaced. This requires the manipulation of culture media which may result in contaminations. Moreover, the sequence of consumption and supply results in time-dependent courses wherein the concentrations of the active substance may vary between ineffective and toxic values. (3) Usually, the resistance gene is of prokaryotic origin. Sections differing from eukaryotic base sequences within the gene and primary sequence patterns within the protein may be recognized by the cell as foreign and contribute to the inactivation of the foreign gene. An inhibition of the expression by neomycin phosphotransferase was demonstrated in retroviral systems (Artelt, P. et al., Gene 99:249-254 (1991)). In still other systems a disturbance of RNA splicing by aminoglycosidic antibiotics could be demonstrated (Waldsich, C. et al., RNA 4:1653-63 (1998)). (4) Antibiotics are expensive and environmentally polluting. (5) Steps for the separation and inactivation of antibiotics have to be scheduled in the product purification (Panayotatos, N., Gene 74:357-63 (1988)).

An eukaryotic selection system evading some of these problems is based on the expression or recombinant glutamine synthetase (Barnes, L. M. et al., Cytotechnology 32:109-123 (2000)). This enzyme catalyzes the glutamine synthesis by amidating glutamates with ammonium ions. Some cell lines have lost this enzyme and can therefore only survive in media supplemented with glutamine. For these cells a selection in media without added glutamine is suited with the foreign gene being coupled to the expression of glutamine synthetase. However, the deprivation of glutamine from the medium must be performed only gradually since cells expressing recombinant glutamine synthetase will also die if the change to a glutamine-free medium occurs too fast.

Moreover, glutamine synthetase may purposively be used as a so-called amplifiable marker for the production of high-expression cell lines (Barnes, L. M. et al., Cytotechnology 32:109-123 (2000)). This method uses the property of some cells of increasing the expression level of some gene products by multiplying the number of copies of the gene. For a gene amplification of glutamine synthetase gradually increasing concentrations of the specific inhibitor methionine sulfoximine (MSX) are added to the medium. The inhibitor decreases the effective activity of the glutamine synthetase. This is compensated by an increase of the gene dose. Foreign genes closely neighbouring the glutamine synthetase gene may also be multiplied in the thus-induced gene amplification and hence increasingly expressed. The gene amplification may also be successful if the cell has an endogenous glutamine synthetase.

A comparable principle uses the dihydrofolate reductase (DHRF) gene amplification (Banerjee, D. et al., Cancer Gene Ther. 1:181-4 (1994)). This enzyme converts folate via dihydrofolate into tetrahydrofolate, an essential educt for the de novo synthesis of purines, pyrimidines and glycine. DHFR is specifically inhibited by methotrexate (MTX). A gradual MTX addition may enforce an increase of DHFR expression by an amplification of the copy number of the gene. If a foreign gene is inserted into the chromosome too closely neighboured to recombinant DHFR, an amplification of the number of foreign gene copies may be achieved by the addition of MTX via the amplification of recombinant DHFR as well.

Even if gene sections recombinantly inserted in a cell have been stabilized in the chromosome and manipulated to yield the greatest possible expression, the production of active substances in the bioreactor can fail if the cultivation conditions result in the induction of apoptosis. This problem has been known in literature (Frahm, B. et al., Bioprocess Biosyst. Eng. 26:1-10 (2003)) and minimized by interference with the apoptopic program. Proteins of the Bcl 2 gene family are important sensors of nutrient deficiency with some of said proteins acting pro-apoptopically and others such as the prototypical Bcl 2 protein may stop a started apoptopic signal cascade (Gross, A. et al., Genes Dev. 13:1899-911 (1999)). A recombinant overproduction of Bcl 2 has proven to be favourable in the production of active substances (Frahm, B. et al., Bioprocess Biosyst. Eng. 26:1-10 (2003)).

WO 92/21756 discloses the stimulation of insulin production by increasing glucose concentrations. Moreover, the introduction of a glucose carrier gene is disclosed. However, the mechanism is a component of a physiological regulatory loop, a stimulation of the expression of foreign genes by decreasing glucose amounts is not disclosed.

F. Ismail-Beigi and G. Vanderberg, Arch. Biochem. Biophys. 331(2):201-207 (1996) describe the expression of a recombinant GLUT 1 carrier in a liver cell line of rats. Moreover, regulation of the glucose transport as a response to stimuli such as the insulin concentration or oxygen deficiency is discussed and it is shown that a poison of the respiratory chain (sodium azide) simulates the glucose transport. A linkage of the deficiency state with the high expression of a foreign gene and the protection of cells from cell death which is induced by nutrient limitations that not been shown.

Also D.-Y. Hwang and F. Ismail-Beigi, Arch. Biochem. Biophys, 339(2):206-211 (2002) describe the transport of glucose in the presence of a metabolic poison (here: cobalt chloride) simulating oxygen deficiency. The recombinant expression of GLUT-1 shows that in some systems the glucose transport is the rate-determining step in the glucose metabolism. Vice versa, in other systems the glucose metabolism results in an accumulation of lactate if the oxygen deficiency-induced transport causes excessive intracellular glucose concentrations. The possibility of linking the expression of a foreign gene to the expression of the GLUT 1 transporter has not been shown.

T. Asano et al., J. Biol. Chem. 264(6):3416-3420 (1989) describe the stimulation of the expression of a glucose carrier molecule by insulin in HepG2 cells, a human hepatoma line. The addition of insulin to the culture medium results in an increased expression of the glucose carrier and hence also in an increased glucose uptake. A linkage of the expression of a foreign gene to the expression of the GLUT 1 transporter is not mentioned.

N. R. Cohen et al., Biochem. J. 315:971-975 (1996) describe the expression of GLUT 1 from rat in a slime mould, Dictyostelium discoideum. Cultured cells of said eukaryote were transfected with GLUT 1 such that the glucose carrier is stably expressed. Wild-type cells had only very low glucose transport rates. It is demonstrated that GLUT 1 from mammals selectively transports hexoses even across animal kingdom borders, the kinetics being comparable to that in human erythrocytes. It is not investigated nor described whether an increased glucose uptake is associated with advantages for the recombinant cells.

WO 97/15668 describes the use of a therapeutic agent against neoplastic diseases, namely streptozotocin or STZ. This agent is a glucose analogue and enters the cell via a glucose transport mechanism. Several possible applications for STZ and GLUT 2 recombinant cells are proposed, however, a linkage with the expression of a foreign gene are not mentioned.

Hence, there is still a need for a selection system wherein the maintenance and high expression of foreign genes is possible independent of external selection markers and at the same time an induction of apoptosis is prevented, hence not—which would be the case in the Bcl 2 overproduction—an already running program in a predamaged cell is terminated.

### BRIEF DESCRIPTION OF THE INVENTION

Now, it has been found that cells especially suited for the stable expression of foreign genes can be generated by a special manipulation, namely the expression of sugar carrier proteins which improve the energy metabolism of cells and in particular permit an improved usage of sugars despite a depletion of nutrients or cofactors in the medium which may be purposive or induced by technical requirements. In a preferred variation of the invention the cell cultures are protected from necrosis and/or the induction of apoptosis under adverse growth conditions, e.g., at a very high cell density in bioreactors or in the implantation as cell capsules in an organism.

Some cell lines such as, e.g., CHO cells react with apoptosis to the above-mentioned deficiency conditions, others, such as, e.g., HEK 293 cells, slow down proliferation (Zhangi, J. A. et al., Biotechnol. Bioeng. 64:108-119 (1999) and the author's own observations). The 293 cells will briefly be discussed in connection with FIG. 5. However, cells now expressing the sugar carrier protein are better suited to use the low nutrient concentration in the culture supernatant and can proliferate.

Moreover, it is surprising that the culture itself gradually adjusts the conditions resulting in an increased selection pressure towards highly expressing cells by the consumption of sugars. Moreover, it is not necessary to add antibiotics such as gentamycin or hygromycin B to the nutrient medium in order to ensure the maintenance of foreign genes.

Surprisingly, it was observed in the development of the system that the production rates of foreign proteins in cells were increased under, e.g., glucose deficiency conditions. However, since the proliferation rate correlated with the glucose supply, the specific production (production rate per cell) under glucose deficiency was increased and the ratio of secreted foreign protein to other components in the culture supernatant was shifted towards an advantageous purification. It is supposed that in an early state of cell stress induced by glucose deficiency the extensive activation of stress factors also results in a metabolic activation of the cell and thus in increased expression levels for the foreign protein. However, a sustained glucose deficiency induces the unfolded protein response (UPR; i.e., the transcriptional activation of genes due to the accumulation of misfolded proteins) and, moreover, is the reason for an incorrect processing of glycoproteins in the endoplasmatic reticulum and the Golgi apparatus (Kaufman, R. J. et al., Nat Rev Mol Cell Biol 3:411-21 (2002)). The system tested and described herein possibly inhibits the induction of the massive stress response and enables the synthesis of normally processed glycoproteins also with reduced glucose concentrations. These properties are an important advantage in the production of recombinant proteins and pharmaceutically active substances.

Low glucose values also interfere with the replication of some viruses, e.g., adenoviruses (Bardell, D., Microbios 20:139-44 (1977)), which have an important medical significance as viral vectors or for the production of vaccines. Moreover, the increased glycolysis in the infected cells results in a faster depletion of glucose in the medium (Bardell, D., Microbios 20:139-44 (1977)). However, since glucose is toxic for the host cell in higher concentrations (Acker, H. et al., Cancer Res 47:3504-8 (1987); Medina-Navarro, R. et al., Hum Exp Toxicol 23:101-5 (2004)), the increased glucose demand can be compensated by an increased glucose addition only in a limited range. The system described herein facilitates the usage of sugars even at decreased concentrations in the culture medium and thus enables improved yields in the pharmaceutical production of viruses or viral vectors.

The protection of cells from apoptosis and/or necrosis in a high-density reactor follows a principle related to selection. However, in this case the system is primarily meant to buffer local variations of the nutrient concentrations. In this manner the culture is protected from necrotic and apoptotic foci in regions of lower diffusion which increases the culture time and production intervals and decrease the proportion of metabolites.

Moreover, since the expression of foreign genes requires energy, it is surprising that the glucose limitation in the present invention results in an increased expression of a foreign gene, because the glucose limitation in the extracellular space inhibits the passive influx of glucose.

Hence, the invention pertains to:

(1) a process for the expression of one or several foreign genes in eukaryote cells (hereinafter briefly “cells”) comprising the support of the energy metabolism of said cells by introducing into the initial cells a functional nucleic acid segment coding for a sugar carrier gene, the expression of which improves the energy metabolism of the transformed cells as compared to the initial cells (if it is expressed in the cells) and the expression of which is linked with the expression of foreign genes;

(2) a process for the selection of an eukaryote cell, said cell being suitable for the expression of one or several foreign genes, wherein the selected cell has been modified by introducing a functional nucleic acid segment coding for a sugar carrier protein, the expression of which improves the energy metabolism of the cells as compared to the initial cells and the expression of which is linked with the expression of foreign genes, and which is suited in particular in carrying out a process according to (1) and wherein the process comprises one or several of the steps

(a) transformation or transfection of initial cells by said functional nucleic acid segment and

(b) selection via depletion of one or several nutrients or cofactors transported by the sugar carrier gene;

(3) eukaryote cells which are suited for a process defined in (1) and/or obtainable by the process according to (2) and which are transformed or transfected by a functional nucleic acid segment as defined in (1);

(4) a functional nucleic acid segment defined in (1) and

(5) the use of cells defined in (3) for the expression of foreign genes and the multiplication of viruses.

### DESCRIPTION OF FIGURES

FIG. 1: CHO cells transfected with pIJO-22-GlutGFP (SEQ ID NO:1). Representative sections of the culture at day 7, 14 and 15 after transfection (a, b; c, d and e, f, resp.) are shown. For every time value phase-contrast micrographs (a, c, e) and fluorescence micrographs for the detection of GFP are shown. Cell death induction by glucose deficiency is evident from day 14. The dynamic of this process becomes apparent by comparing the cell density on the two following days. Cells carrying the GLUT-GFP cassette preferably survive.

FIG. 2: parallel control experiment to 1: CHO cells transfected with pIJO-25-NeoGFP (SEQ ID NO:2). Representative sections of the culture at day 7, 14 and 15 after transfection (a, b; c, d and e, f, resp.) are shown. For every time value phase-contrast micrographs (a, c, e) and fluorescence micrographs (b, d, f) for the detection of GFP are shown. From day 14 a uniform dying of the culture independent of the presence of the transfected episome becomes apparent. The transfection efficiencies for pIJO-22-Glut-GFP (SEQ ID NO:1) in FIG. 1 and for pIJO-25-NeoGFP (SEQ ID NO:2) in FIG. 2 were identical.

FIG. 3: CHO cells transfected with pIJO-22-Glut-GFP (SEQ ID NO:1) (a, b) or pIJO-25-NeoGFP (SEQ ID NO:2) control plasmid (c, d) were preselected with hygromycin B and cultivated without changing the medium until the cultures had died to a confluency of about 5%. Subsequently, the culture was cultivated to confluency by regular medium changes and photographed under phase contrast (a, c) and under a fluorescence filter to detect the reporter protein (b, d). Due to the adverse cultivation conditions, subpopulations lacking the episome could form in the preselected pool. This effect was distinctly marked in the transfection with the reference plasmid (Neo-GFP cassette; SEQ ID NO:2), but not with the effector plasmid (GlutGFP cassette; SEQ ID NO:1).

FIG. 4: marked expression increase of selected alkaline phosphatase (SEAP) in the vector pIJO-76 (GLUT-PV-SEAP) (SEQ ID NO:3) in minimal medium. At the end point of measurement, the SEAP activity is 6 times higher as compared to the reference (pIJO-73 (PV-SEAP) (SEQ ID NO:4) in minimal medium). Also in other media (complete medium or complete medium with 600 μg/ml of the G418 selection reagent) the expression of pIJO-76 (GLUT-PV-SEAP) (SEQ ID NO:3) is better than that of the reference plasmid pIJO 73 (PV-SEAP). Solid symbols for experiments with the effector plasmid pIJO-76 (GLUT-PV-SEAP) (SEQ ID NO:3), outline symbols for the reference plasmid pIJO 73 (PV-SEAP); rhombi for a cultivation in complete medium, squares for a complete medium in combination with G418, triangles for a minimal medium.

FIG. 5: 293 cells with plasmid with an episomal GLUT-IRES-GFP cassette (pIJO-71 SEQ ID NO:5) grow in an unsupplemented medium and with glucose deficiency better than wild type cells. Wild type cells are shown in the left hand column and transfected cells are shown in both of the right hand columns. The transfected cells were selected with G418 for 8 weeks prior to the start of this experiment. The unsupplemented glucose deficiency medium M4566 was not changed for the duration of the experiment (figures a-i). Identical starting conditions are shown in a-c. The advantage for the transfectants becomes apparent after 4 weeks of cultivation (compare g with h and i), but not after 2 weeks (d-f). Reference in figures j-l: cell densities for the wild type and the transfectant in unsupplemented F10 medium.

### DETAILED DESCRIPTION OF THE INVENTION

In the process according to embodiment (1) of the invention, a functional nucleic acid segment is expressed in the cells and thus the energy metabolism of the cells as compared to the initial cells is improved.

In the context of the present invention, in particular within the meaning of embodiment (4) of the invention, a functional nucleic acid segment codes for a sugar carrier gene, preferably for a factor improving the utilization of mono, di- or oligosaccharides by the transformed cells as compared to the initial cells. Here, the supported energy metabolic pathway is preferably selected from glycolysis, citrate cycle, pentose phosphate pathway, gluconeogenesis, with glycolysis being the preferred energy metabolic pathway. The nucleic acid segment of the invention may be a DNA segment (contained in a vector, plasmid etc.) or a RNA segment (viral RNA vector). In addition to the sequence coding for the sugar carrier protein the functional nucleic acid segment may also comprise other functional sequences required for a successful transformation and/or expression, preferably promoters, foreign genes to be expressed, IRES elements, selection markers, activator proteins of regulatory cascades, cis and trans active factors for the improved translation of an mRNA or for the recognition by viral factors, e.g., for the packaging into envelops or for replication etc.

In the context of the present invention, a “nutrient” is a compound which is converted by the primary metabolism of a cell for the generation of energy. Sugars are preferred nutrients in carrying out the present invention.

Hereinafter, native sugars and the derivatives thereof (“sugar derivatives”) will be understood as sugars. Preferably they are mono-, di- or oligosaccharides, particularly preferably monosaccharides, especially preferably hexoses. Of the latter, in particular glucose and fructose and the corresponding pyranosides and furanosides are preferred. Glucose is exceptionally preferred. Sugar derivatives within the meaning of the present invention are compounds formed by derivatization of functional groups of native sugars, e.g., alkylated and acylated sugars, but also reduced or oxidized sugars, e.g., sugar alcohols such as sorbitol or xylitol.

A “sugar carrier protein” is a protein which is suited for the transport of one or several sugars. It is coded by a sugar carrier protein gene. Hexose carrier proteins are preferably used in carrying out the process according to (1) or (2).

A “depletion” within the meaning of the present application is a deficiency in specific nutrients in the medium, in particular in specific sugars as compared to the concentration optimal for the growth and/or the obtainment of cells. Thus, a sugar depletion is characterized by a concentration of up to 1 g/l, preferably of up to 0.5 g/l, preferably of up to 0.2 g/l of the sugar which is transported by the sugar carrier protein and the concentration of which therefore has a direct influence on the metabolism of the transformed cell at low concentrations of alternative nutrients in the fresh medium. A low concentration of alternative nutrient means a concentration not being increased as compared to usual concentrations of these nutrients in standard media. A low concentration of alternative nutrients is provided if—in the case of glucose as transported hexose—no other hexoses are contained in the medium in addition to glucose at the mentioned sugar concentrations. Hence, in the medium sodium pyruvate should preferably have a concentration of maximal 200 mg/l, in particular of maximal 110 mg/l, glutamine should preferably have a concentration of maximal 4 mM, in particular of 2 mM, and glutamate should preferably have a concentration of maximal 120 mg/l, preferably of maximal 80 mg/l.

In the process of the invention eukaryote cells (“cells”), i.e. vertebrate, invertebrate or plant cells, may be used as initial cells with vertebrate or invertebrate cells being preferred. Mammalian cells (such as rodent, canine, swine, bovine or primate cells (including human cells)) are especially preferred. Also cells of lower eukaryotes (such as yeasts and schizomycetes) may be used. The term “cells” within the meaning of the present invention also comprises tissue/tissue cultures and (non-human) organisms made of the above-defined cells. Primary or transformed cells may be used in the process of the invention. Here, preferred cells comprise human fibroblasts, CHO cells, BHK cells, NSO cells and HEK-293 cells.

Preferably, the process according to (1) or (2) comprises the cultivation of an above-defined transformed eukaryote cell and the isolation of the expression product from the culture. The cultivation within the scope of processes (1) and (2) may be performed in vitro or in vivo. However, it will preferably be performed in vitro. An unexpectedly beneficial effect of the processes according to (1) and (2) is that at a given day the cell density of cultures in a minimal medium is distinctly less than the density of the cells in the complete medium, although the expression level of the genes is not essentially different (observation in example 2C). Hence, this results in a higher specific expression activity of a single cell in the minimal medium. This effect offers an important advantage in production since the depletion of secreted metabolites in the purification of the product from the culture supernatant is easier.

In the processes (1) and (2) of the invention, the functional nucleic acid segment may transiently or stably be inserted in the cells. This is performed by processes known to the skilled person such as the transformation and transfection with suitable constructs (vectors, plasmids etc.) which comprise the above-defined functional nucleid acid segment.

In a preferred aspect of the process (1), the sugar carrier protein is a hexose carrier protein, especially preferred it is a glucose carrier protein. The latter is either a passive glucose carrier protein facilitating the diffusion of glucose, fructose and/or other hexoses into the cells, or an active glucose carrier protein transporting glucose, fructose and/or other hexoses by an active symport system into the cell. Preferably it is a passive glucose carrier protein selected from the group comprising GLUT-1, GLUT-2, GLUT-3, GLUT-4, GLUT-5 or GLUT-7 proteins. Exceptionally preferred the sugar carrier protein is GLUT-1.

In another aspect of the process (1) according to the invention, the initial cell has at least one copy of the gene coding the sugar carrier protein. In an alternative embodiment, the gene is inactivated in the initial cells. The system described herein will also work in cells (CHO and HEK 293) having intact endogenous metabolic pathways which are complemented by the system according to the invention.

The sugar carrier protein gene in process (1) or (2) may be heterologous or homologous to the initial cell. However, with a heterologous sugar carrier protein one has to consider that said protein will fulfil the desired function in the transformed cell. The sugar carrier protein of the present invention preferably originates from eukaryotes, more preferred from mammals, especially preferred from human or mice. Especially preferred is a human sugar carrier protein.

In processes according to embodiment (1), the expression of the sugar carrier protein is linked to the expression of a foreign gene. This linkage will be formed either by a direct linkage of the foreign gene expression with the expression of the sugar carrier protein gene or by a regulatory cascade. The direct linkage is possible due to the control of the expression of both genes by a common promoter. If this promoter is bidirectional, discrete transcripts may be formed for both genes. However, in a preferred, especially close linkage both transcripts are located on one bicistronic RNA molecule. In an especially preferred application, the expression of the gene in the rear position is enabled by an IRES element linking both genes with each other. Preferred IRES elements are those of the encephalomyocarditis virus (Ghattas, I. R. et al., Mol Cell Biol 11(12):5848-59 (1991)) or the polio virus (Hailer, A. A. et al., J Virol 67(12):7461-71 (1993)). Leader sequences of specific cellular genes such as those of the myc gene permitting a cap-independent translation initiation are also suitable as IRES. After alternative splicing, both genes may also be translated by one primary transcript.

Regulatory cascades which are able to control the expression of a gene are suited for linking the expression of the sugar carrier protein gene with expression of the foreign gene. Preferably, in such a cascade the expression of the sugar carrier protein gene is directly linked with the expression of an activator protein, preferably according to the above description, wherein the activator protein in turn activates the expression of the foreign gene at another DNA site. Activator proteins suited for regulatory cascades are, e.g., steroid receptors. In this variant the foreign gene may stably be transfected or transiently incorporated in the cell with a stable transfection being preferred.

There are no restrictions as to the foreign genes which may be expressed according to the present processes. This comprises pharmacologically effective proteins such as, e.g., cytokines, hormones, receptors, membrane-bound or soluble antibodies and fusion proteins, at least one of which being a partner of one of the above pharmacologically effective proteins.

In processes according to (1) or (2), the transient expression of proteins or the stable integration of plasmids or gene sections is desired. In the latter case, episomal plasmids are not necessary any more. A stable or transient viral vector system, e.g., a retroviral or alphaviral vector, may also be used to insert the functional nucleic acid fragment.

In addition to the signal elements to control the foreign gene expression and the sugar carrier protein gene (i.e., the promoter(s), enhancer and polyA), a suitable vector for carrying out the process according to (1) or (2) comprises at least one ori element (a transcription start element active in mammalian cells) and at least one element producing an association with the chromosomal matrix and a distribution during cell division, and in addition it comprises other sequences ensuring the multiplication of the vector in multiplication host cells, hence, in particular in bacteria. Preferably, such a vector furthermore contains at least one gene for a classic selection marker (e.g., neomycin phosphotransferase (npt), hygromycin B-phosphotransferase (hpt), blasticidin deaminase (bda) and puromycin N-acetyl transferase (pac)). A plasmid preferably used for transformation provides cis and trans active factors for an obtainment as episome. Thereby, the stable expression of the foreign gene can be achieved without an integration into the genome of the initial cell. In a preferred aspect of the process, the cells are initially selected with one or several antibiotics. Subsequent to the formation of a stable cell line, the cells are kept under glucose deficiency conditions without adding antibiotics. Especially suited for carrying out the process according to (1) or (2) is pREP-4 (Invitrogen, San Diego, U.S.A.).

In further aspects of process (1) the expression of foreign gene and sugar carrier protein is provided by a dependent expression of two promoters on the same plasmid backbone or by a dependent expression by a cotransfection with different plasmids. Therein, the dependency is created by the spatial proximity and flanking genomic sequences. In other aspects the common expression is achieved by the formation of chimeric, not further processing proteins or by fusion proteins which are cotranslationally (e.g., by a ribosomal reading frame change) or posttranslationally, e.g., by proteolysis, separated from each other.

Preferably, in the process according to (1) or (2) the culturability, in particular the surviving and the proliferation of the recombinant cell population is purposively supported by regulating the amount of nutrients, in particular of sugar, in the culture medium.

In another preferred aspect of process (1) the process comprises a preselection using a conventional selection principle. This is preferably performed by adding an inhibitor of the recombinant or endogenous sugar carrier protein for the selection of the gene amplification. Due to the linkage of the expression of the sugar carrier protein with the expression of a foreign gene coding for a product, the specific yield of the foreign gene product may alternatively be increased by a purposive or process-related nutrient depletion, the cells being protected by the recombinant sugar carrier and in particular from apoptosis, necrosis or proliferation inhibition.

In additional preferred aspects of the processes according to (1) or (2), cultivation is performed only under glucose depletion conditions in order to enrich transfected or transduced cells without adding antibiotics. Alternatively, a simultaneous or alternating selection with antibiotics and glucose depletion is possible, wherein the alternating selection may be performed in various combinations and time intervals.

In further aspects of the processes (1) and (2) the selection and/or the maintenance of foreign genes is supported by the application of stress. Stressors may be, e.g., inhibitors of mitochondrial processes or variations of the insulin addition. Moreover, the usability of nutrients is modulated by the adjustment of ion concentrations in the culture medium (particularly of the ions Na⁺ and K⁺ which are involved in the active symport).

According to the inventive process, the use of cells in the bioreactor, in tissue or in the organism can be designed such that a process-related nutrient depletion is buffered better than by the initial cells.

Moreover, in a preferred aspect of (1) the transformed cells obtained by the inventive process (1) are suited as a host for the selective multiplication of viruses or viral vectors.

In step (a), the selection process according to (2) preferably comprises an additional preselection using a conventional selection principle, in particular the preselection with antibiotics, primarily with neomycin or hygromycin. Furthermore or alternatively, it preferably comprises one or several of the process steps of the process according to (1) and/or it is performed according to the description of the preferred aspects of process (1). In the selection process according to (2), the functional nucleic acid segment and the sugar carrier protein preferably have one or more of the properties which have above been described for the functional nucleic acid segment in the process according to (1); in particular the sugar carrier protein preferably is a hexose carrier protein, especially preferred one of the above-defined glucose carrier proteins.

The use of relevant knock-out mutants can enhance the selection effect in the process according to (2). A knock-out mutation of the active hexose carrier SGLT-1 was simulated by a low sodium concentration and the absence of pyruvate as alternative energy donor in the medium (example 3).

Hereinafter, the present invention will be illustrated in more detail by the preferred embodiments of (1) and (2), namely with a hexose carrier as a sugar carrier protein. Especially preferred, this hexose carrier is a glucose carrier.

Being a polar molecule, glucose cannot diffuse freely through the plasma membrane, but enters the interior of the cell by receptor-mediated energy-dependent pathways. The system presented here modulates the first and rate-determining step in the use of glucose as energy carrier by supporting the translocation into the interior of the cell by special, recombinant expressed carrier proteins. In this way, very low outer glucose concentrations can be used by the recombinant cells, whereas wild type cells reduce their division rate or even, such as, e.g., CHO cells, react with apoptosis (Zanghi, J. A. et al., Biotechnol. Bioeng. 64:108-19 (1999)). Further advantages of this system are that—contrary to many present selection markers of prokaryotic origin—an eukaryotic gene of the same species may be introduced into the cell. Moreover, with the use of the system described herein an addition of active substances such as antibiotics is not necessary.

Mammalian cells are provided with two mechanistically different carrier systems for glucose and other hexoses, the facilitated diffusion (by passive glucose carrier proteins) and the active symport (by active glucose carrier proteins):

A facilitated diffusion is catalyzed by specific receptors of the GLUT protein family (GLUT1 to GLUT5 and GLUT7) and permits the influx of hexoses (in the case of GLUT5 primarily of fructose in addition to glucose) along a concentration gradient (Brown, G. K., J. Inherit. Metab. Dis. 23:237-46 (2000)). In the cytoplasm, the gradient is deprived from the hexoses by phosphorylation. Thus a back pressure is avoided. GLUT1 to GLUT 5 are integral proteins of the plasma membrane, GLUT7 is expressed on the intracellular membranes of the endoplasmatic reticulum.

Active carriers bring metabolites such as amino acids and sugars into the cytoplasm using electrochemical gradients. In this very heterogeneous super family of proteins, the glucose transport is performed by SGLT1 (Wright, E. M. et al., J. Exp. Biol. 196:197-212 (1994)) which uses the Na⁺ concentration gradient in a so-called symport for the active enrichment of glucose in the cell. In turn, the driving Na⁺ gradient is maintained by hydrolysis of ATP in the antiport of Na⁺ in the lumen against K⁺ from extracellular space.

In a preferred embodiment of process (1) the foreign gene and the sugar carrier protein gene are linked to each other on an mRNA by an IRES element. Preferably, the sugar carrier protein gene codes for a glucose carrier. The glucose carrier may be an active carrier or a factor facilitating the diffusion of nutrients.

Not only glucose carrier proteins may be used for the process according to (1) or (2): in another preferred aspect of the invention the fructose-specific carrier GLUT5 is expressed, and the system is used by employing fructose as energy carrier. In still other aspects a combination of carriers, e.g., of GLUT1 and GLUT5, of GLUT1 and GLUT7 (which is present in the endoplasmatic reticulum), or of SGLT1 and GLUT1 and/or a combination of sugar molecules or also of sugar derivatives is used. In a combination of substances, e.g., glucose is used for the selection of SGLT1 recombinant cells and genistein (Vera, J. C. et al., J. Biol. Chem. 271:8719-8724 (1996)) is used in order to inhibit endogenous GLUT1 carriers.

The system presented herein uses a recombinant glucose carrier in eukaryote cells in two fields of application: (i) the expression of the carrier is linked to the expression of a foreign gene and thus enables a selection pressure ensuring the maintenance of the foreign gene, or (ii) the carrier may also be expressed independently of a foreign gene to enable a selection of the cells and/or improve the culturability of cells or the product yield under adverse growth conditions.

In further aspects the principle of the system is used with or without an additional expression of a carrier at later stages in the energy metabolism of the cell. Thus, e.g., glucohexokinase in the cytoplasm or aconitase in mitochondrions, the site of the citrate cycle, may be modulated in their expression by recombinant methods or their activity by a chemical or biological induction.

In further aspects changes of the nutrient concentration and/or specific inhibitors of the carriers and/or substances having chromosome-destabilizing properties are used for the gene amplification of a foreign gene, the expression of which being linked to the expression of the carrier. In one embodiment of this aspect specific active substances are used to inhibit endogenous transport processes. Hence, the use of the recombinant carrier is intensified. In again other aspects there are used cells, the endogenous hexose carrier molecules of which are inactivated for example by antisense or knock-out technology.

In another embodiment the carrier or a combination of carrier and foreign gene is expressed in cells in order to provide said cells with a better culturability or an improved surviving under adverse conditions. These cells may for example be employed under high cell density conditions in a bioreactor.

In one aspect of the invention the cell provided with the carrier may be destined for the use in a living organism, e.g., for the ex-vivo gene therapy or for the implantation of a cell assembly or a cell capsule. The expression of the carrier can create a cell being more favourable for the application also in sites of low nutrient concentrations or diffusion.

The invention will be illustrated in more detail by the following examples which, however, do not limit the scope of the invention.

### EXAMPLES

### Example 1

### Design of the GLUT-1 System

Total RNA was extracted with acidic phenol from HeLa cells and transcribed with dT-16mer primers by SuperScript II Reverse Transkriptase (Invitrogen, Carlsberg, Calif., USA) in cDNA. Due to a surprisingly low yield of PCR amplification products the cDNA was pre-amplified with the primers i104 (SEQ ID NO:10) and i105 (SEQ ID NO:11), and this PCR reaction was used in another PCR reaction with the actual cloning primers i100 (SEQ ID NO:6) and i101 (SEQ ID NO:7). The amplifications and clonings were planned using the gene bank sequences #NM006516 and #XM002033 (which differ from each other in three regions). For a directed cloning into the episomal vector pREP4 (Invitrogen, Carlsbad Calif., USA) the primers i100 (SEQ ID NO:6) and i101 (SEQ ID NO:7) contain unique restrictase target sequences (HindIII and XhoI). Moreover, the vector pREP4 expresses the resistance factor against hygromycin B.

In order to be able to trace the obtainment of transfected episomal plasmids, an EMCV-IRES-based expression cassette for the EGFP reporter protein (Clontech, Palo Alto Calif., USA) was inserted immediately behind the open reading frame for GLUT1, whereby the plasmid pIJO-221-GLutGFP (SEQ ID NO:1) was formed.

### Example 2

### Use of the GLUT-1 System

A. Selection: As a control for a successful enrichment of pIJO-221-Glut-GFP (SEQ ID NO:1) due to the expression of GLUT 1 the reading frame for GLUT1 was substituted by the gene for the eukaryotic selection marker neomycin phosphotransferase (Neo) by opening pIJO-22 with HindIII and XhoI and inserting Neo as StuI and BstBI fragment from pEGFP-N1 (Clontech) after a treatment with Klenow DNA polymerase using T4 DNA ligase, whereby the plasmid pIJO-25-NeoGFP (SEQ ID NO:2) was formed. The skilled person knows that Neo is nontoxic and that differences in the enrichment of pIJO-221-GlutGFP (SEQ ID NO:1) and pIJO-25-NeoGFP (SEQ ID NO:2) hence should be assignable to the presence of GLUT1.

The plasmids pIJO-221-GlutGFP (SEQ ID NO:1) and pIJO-25-NeoGFP (SEQ ID NO:2) were transfected into CHO cells as a liposomal complex, and the expression of the GFP marker gene was observed for 15 days. In this and other experiments transfection was effected by LipofectAMINE Plus (Invitrogen): on the day before transfection, CHO cells were seeded in 12-well plates. For transfection, 750 ng plasmid DNA were mixed with 50 μl Opti-MEM (Invitrogen) and 5 μl Plus Reagent, incubated at room temperature, and after 15 min 2 μl LipofectAMINE in 50 μl Opti-MEM were added. After additional 15 min the culture medium was replaced by fresh 400 μl Opti-MEM mixed with the transfection mix. After 3 h 1 ml medium was added.

Early experiments showed that the serum-free Opti-MEM recommended for liposomal transfections contains too much glucose, which results in an only slow increase of a selection pressure due to the reaction of glucose. Therefore, in further experiments the transfection protocol was adjusted to a transfection in a glucose-reduced medium. In the experiments described herein, glucose-reduced medium (Sigma Taufkirchen, D) was MEM Medium #M4655 (Sigma Taufkirchen, D) supplemented with 5% calf serum. MEM #M4655 is characterized by relatively low glucose (1 g/l) and NaCl (6.8 g/l) concentrations; due to the novel nature of the present system, a medium having still lower glucose concentrations could not be found. Pyruvate is not added to this medium. Pyruvate is the very energy-rich product of glycolysis and ultimately converted to energy in the subsequent citrate cycle. Due to the removal of pyruvate the cells are forced to transport glucose into the cells in order to produce energy.

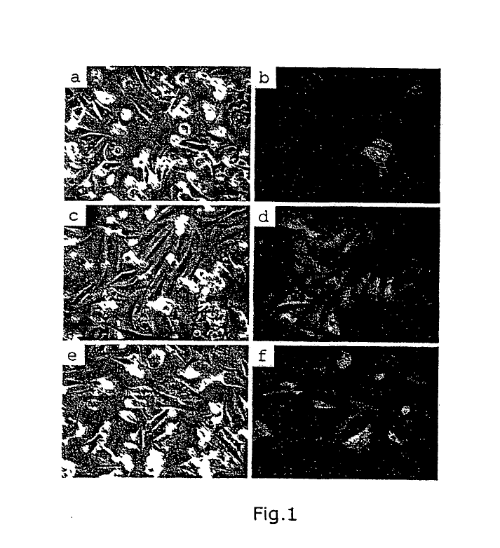

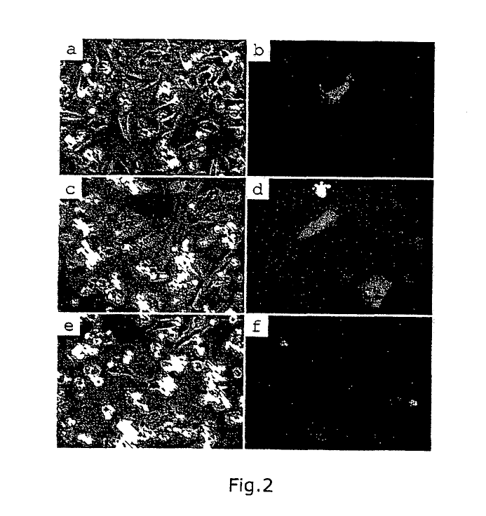

FIGS. 1 and 2 show the time-dependent course of the GFP expression in CHO cultures which were transfected with pIJO-221-GLutGFP (SEQ ID NO:1) (FIG. 1) or the control plasmid pIJO-25-NeoGFP (SEQ ID NO:2) (FIG. 2) in parallel. The cultivation of both assays was performed in MEM #M4655. After initially comparable numbers of GFP-expressing cells and comparable signal intensities of the reporter protein for both constructs, only in the culture transfected with pIJO-221-GlutGFP (SEQ ID NO:1) a marked increase of GFP-positive cells takes place. Moreover, it is found that the cells expressing GLUT-GFP suffer from cell death in the depletion medium to a remarkably lower extent than the Neo-GFP controls.

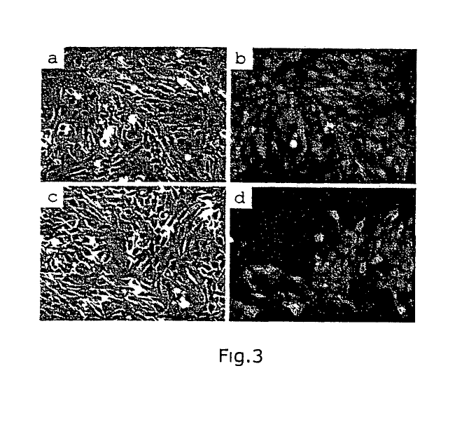

B. Maintenance of the episome under stress: for FIG. 3, CHO cells transfected with pIJO-221-GlutGFP (SEQ ID NO:1) and pIJO-25-NeoGFP (SEQ ID NO:2) were enriched for 20 days by conventional selection with 200 μg/ml hygromycin B, 6-fold diluted, transferred in T25 culture bottles under MEM #M4655 and subsequently cultured for 20 days without any medium change.

These extremely adverse culturing conditions resulted in a massive decrease of viable cells to a confluency of about 5% and might resemble the limiting conditions within a reactor.

Subsequent to this treatment, regular medium changes against MEM #M4655 lacking hygromycin were performed (for additional 14 days) until confluency was attained. FIG. 3 shows a distinct enrichment of GlutGFP-positive cells as compared to the cells expressing Neo-GFP.

C. Quantification: The GFP reporter gene permitted a surprisingly distinct confirmation of the inventive function of the system. Hereinafter, expression results for another reporter, secreted alkaline phosphatase (SEAP), will be quantified. To this end, an expression cassette for SEAP was inserted downstream to the ORF for Glut in an episomal vector. Since the original amino-terminal sequences may be required for the secretion of SEAP, the polio virus (PV) IRES element was used as the IRES element. Said element differs from EMCV-IRES in that the IRES does not have to provide an ATG initiator codon. The episomal vector with the Glut-PV IRES-SEAP cassette was designated as pIJO-76 (SEQ ID NO:3), and it was formed by an insertion of the Glut gene from pIJO-62 (SEQ ID NO:12) as a HindIII fragment having 1602 bp in the vector pIJO-7i3 (SEQ ID NO:4). pIJO-62 is an intermediate clone containing a PCR amplification product (with the primers i100 (SEQ ID NO:6) and i101 (SEQ ID NO:7)) of the Glut gene; PIJO-73 (SEQ ID NO:4) is an EBV-episomal vector containing a PV-IRES-SEAP cassette behind a multiple cloning site; this cassette was formed by an insertion of SEAP as HindIII (Klenow-treated) NotI fragment from pIJO-53 (SEQ ID NO:13) in the vector pBS polio opened with SmaI and NotI. In the experiments with SEAP, the original episomal vector (pIJO-73 (SEQ ID NO:4) lacking the ORF for Glut without substitution serves as a negative control. The episomal vector used for the SEAP experiments carries the resistance gene for the inactivation of G418 (Neo, neomycin phosphotransferase).

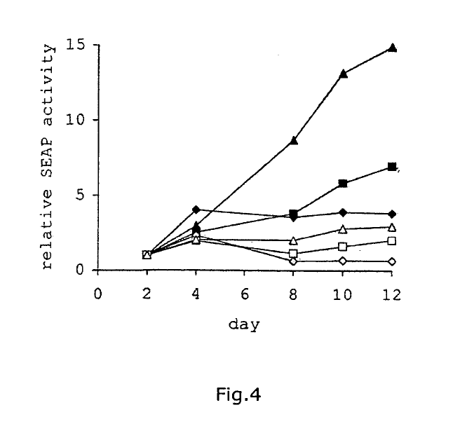

CHO cells were transfected with pIJO-76 (GLUT-PV-SEAP) (SEQ ID NO:3) and pIJO-73 (PV-SEAP) (SEQ ID NO:4), and the secretion of SEAP in the medium was observed for 2 weeks (FIG. 4). Different expression levels between pIJO-76 (GLUT-PV-SEAP) (SEQ ID NO:3) and pIJO-73 (PV-SEAP) (SEQ ID NO:4) were compensated by normalizing the data to the respective first value (day 2): in the control vector pIJO-73 (PV-SEAP) (SEQ ID NO:4) the expression of SEAP in the control vector pIJO-73 (PV-SEAP (SEQ ID NO:4) is higher than in pIJO-76 (GLUT-PV-SEAP) since the SEAP in the reference plasmid does not have an upstream gene. On day 12 the background-subtracted, absolute SEAP value of the illustrated experiment is 667,889 relative light units for pIJO 76 (GLUT-PV-SEAP) in the minimal medium, 546,866 relative light units for pIJO 73 (PV-SEAP) in the complete medium/G418 and 344,356 for pIJO73 (PV-SEAP) in the minimal medium.

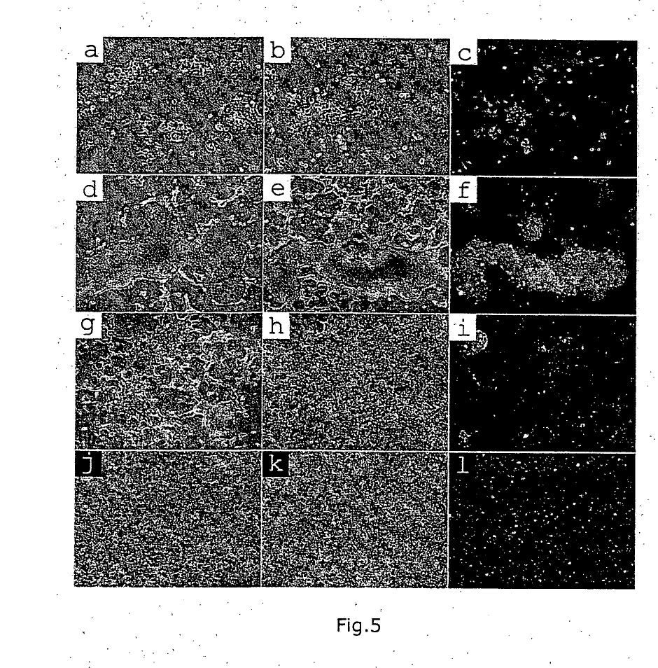

D. 293 cells: HEK 293 cells turned out to be surprisingly insensitive against a glucose limitation. Although these prerequisites are unfavourable for a selection, the Glut system was also inserted in HEK 293 cells and yielded an unforeseen effect: HEK 293 cells were transfected with pIJO-71 (EBN-GLUT-GFP) (SEQ ID NO:5) and selected with G418 for 8 weeks. The stable transfectants were provided in parallel with wild type HEK 293 as a reference to the same confluency in various media (without G418): unsupplemented #M4655, unsupplemented F10 medium and serum-supplemented PBG 1.0 medium. In the following 4 weeks the culture medium was neither changed nor provided with fresh medium. FIG. 5 shows the initial point and two momentary micrographs (after 2 and 4 weeks). Surprisingly, only with glucose deficiency after 4 weeks, but not after 2 weeks a marked difference in the cell density was observed. (With 1100 mg/l the F10 medium also contains little glucose, which, however, is compensated by 110 mg/l sodium pyruvate). The fluorescence micrograph shows that the episomal transgene had completely been maintained over 4 weeks without further G418 selection.

Therefore, the present invention is unexpectedly suited for the support of a long-term cultivation of cell lines which apparently do not react on glucose deficiency.

### Example 3

### Provision of the SGLT-1 System

Complete RNA was extracted from HeLa cells with acidic phenol and transcribed into cDNA with dT-16mer primers by SuperScript II reverse transcriptase. The amplification with the primer pair i102 (SEQ ID NO:8) and i103 (SEQ ID NO:9) and clonings were planned using the gene bank sequence #M24847. For a directed cloning into the episomal vector pREP4, the primers i102 (SEQ ID NO:8) and i103 (SEQ ID NO:9) contain unique restrictase target sequences.

Since initial amplifications by RT-PCR were not successful, HeLa cells cultivated under glucose deficiency were used for further experiments. In this manner a possible induction of the SGLT1 expression, and therewith a relative enrichment of SGLT1 mRNA, was to be achieved. Indeed, RNA isolated from cells treated in this manner could be transformed in RT-RCR in a surprisingly broad spectrum of amplification products. The product spectrum is characterized by very low yields and several signals which imply products having different sizes also in the region of the expected size of 2013 bp. By cloning amplification products having different sizes, the desired sequence could be isolated and transferred into the episomal plasmid pREP4.

1. A process for the expression of one or several foreign genes in eukaryote cells (cells) comprising the support of the energy metabolism of said cells by introducing into the initial cells a functional nucleic acid segment coding for a sugar carrier gene, the expression of which improves the energy metabolism of the transformed cells as compared to the initial cells and the expression of which is linked to the foreign gene expression directly or by a regulatory cascade, wherein the specific yield of foreign gene products is increased by a purposive or process-related sugar depletion.

2. The process according to claim 1, wherein the recombinant sugar carrier protects the cells from apoptosis, necrosis or proliferation inhibition.

3. The process according to claim 1, wherein
(i) glycolysis is the supported metabolic pathway and/or
(ii) the culturability, in particular the surviving and the proliferation of the recombinant cell population, is purposively supported by the regulation of the amount of sugar in the culture medium and/or
(iii) an inhibitor for the recombinant or endogenous carrier is added in order to select the gene amplification and/or
(iv) cultivation is performed in vitro or in vivo, preferably in vitro.

4. The process according to claim 1, wherein
(i) the expressed sugar carrier gene improves the utilization of mono-, di- or oligosaccharides as compared to the initial cells, and wherein in particular the carrier protein is a hexose carrier protein, preferably a passive glucose carrier protein which facilitates a diffusion of hexose into the cells, or an active glucose carrier gene which transports hexose into the cell by an active symport system, wherein a passive glucose carrier protein selected from a GLUT-1, GLUT-2, GLUT-3, GLUT-4, GLUT-5 and GLUT-7 protein is especially preferred; and/or
(ii) the initial cell has at least one copy of the gene coding the sugar carrier protein or the gene in the initial cell is inactivated and/or
(iii) the cells are vertebrate or invertebrate cells, preferably mammalian cells, which may be both primary and transformed cells, wherein primary human fibroblasts, CHO cells, BHK cells, NSO cells and HEK-293 cells are especially preferred; and/or
(iv) the functional nucleic acid segment is transiently or stably inserted into the cells and/or
(v) the functional nucleic acid segment is a DNA or RNA segment and/or
(vi) the foreign genes are selected from cytokines, hormones, receptors, membrane-bound or soluble antibodies and fusion proteins and/or
(vii) the functional nucleic acid segment has additional functional sequences, preferably promotors, foreign genes to be expressed, IRES elements, selection markers, activator proteins of regulatory cascades, cis and trans active factors for the improved translation of an mRNA or for the recognition by viral factors, in particular for the packaging into envelopes or for replication etc.

5. The process according to claim 1, wherein the expression of the sugar carrier protein gene is linked with the foreign gene expression such that
(i) in the direct linkage the foreign gene and the gene coding the sugar carrier protein are linked to each other by a cotransfection of plasmids which contain these genes and integrate them into the genome, by a configuration on one plasmid or one RNA molecule and preferably by an IRES element, in particular an IRES element of the polio virus or the encephalomyocarditis virus, or wherein the expression of the foreign gene and the expression of the gene coding the sugar carrier gene are preferably linked such that after the transcription the gene transcripts are located on one mRNA molecule and linked to each other, wherein a linkage by IRES elements, in particular by IRES elements of the encephalomyocarditis virus or the polio virus is especially preferred, and
(ii) in the linkage by a regulatory cascade the expression of the sugar carrier protein gene is directly linked to the expression of an activator protein, wherein the activator protein activates the expression of the foreign gene(s) located at another site of the DNA.

6. The process according to claim 1, wherein
(i) the concentration of the sugar transported by the sugar carrier protein in the medium is 1 g/l at the most, preferably 0.5 g/l at the most, wherein preferably the concentration of alternative nutrients in the medium is low and/or
(ii) the sugar transported by the sugar carrier protein is a hexose, preferably glucose or fructose; and/or
(iii) the use of the cells in the bioreactor, in the tissue and in the organism is designed such that a process-related nutrient depletion can be buffered better than by the initial cells and/or
(iv) the transformed cells are adjusted as a host to the purposive multiplication of viruses or viral vectors and/or
(v) the process comprises the cultivation of a transformed eukaryote cell as defined above and the isolation of the expression product from the culture.

7. A process for the selection of an eukaryote cell which is suited for the expression of one or several foreign genes, in particular for carrying out a process according to claim 1, and which was modified by inserting a functional nucleic acid segment coding for a sugar carrier gene, the expression of which improves the energy metabolism of the cells as compared to the initial cells and the expression of which is linked to the foreign gene expression directly or by a regulatory cascade, wherein the process comprises one or several of the steps of
(a) transforming or transfecting initial cells with the functional nucleic acid segment and
(b) selecting by depletion of one or several of the nutrients or cofactors transported by the sugar carrier gene.

8. The process according to claim 7, wherein
(i) step (a) furthermore comprises a preselection with a conventional selection principle and/or
(ii) during the selection the concentration of the sugar transported by the sugar carrier protein in the medium is 1 g/l at the most, preferably 0.5 g/l at the most, wherein the concentration of alternative nutrients in the medium is preferably low, and/or
(iii) the process and/or the functional nucleic acid segment has one or several of the properties defined in claims 2 to 6.

9. Eukaryote cells as defined in claim 7 and/or obtainable by the process according to claim 7.

10. The functional nucleic acid segments defined in claim 1.

11. The use of cells as defined in claim 9 for the expression of foreign genes and for the multiplication of viruses.


SEQUENCE LISTING (13 sequences)

SEQ ID NO: 1 - DNA, 12977 bp, artificial sequence
  description of artificial sequence synthetic construct
gccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcccgggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgcccagtggtcgaccaattctcatgtttgacagcttatcatcgcagatcctgagcttgtatggtgcactctcagtacaatctgctctgctgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgcccagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctgggtaccagctgctagcaagcttccatggagcccagcagcaagaagctgacgggtcgcctcatgctggccgtggggggagcagtgcttggctccctgcagtttggctacaacactggagtcatcaatgccccccagaaggtgatcgaggagttctacaaccagacatgggtccaccgctatggggagagcatcctgcccgccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgttgtgtcgctgtttgtggtggagcgagcaggccggcggaccctgcatctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgactcgaaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaatttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggcctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgactctaga

SEQ ID NO: 2 - DNA, 12374 bp, artificial sequence
  description of artificial sequence synthetic construct
gccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaacagcgtcaacagcgtgccgcagatcccgggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgcccagtggtcgaccaattctcatgtttgacagcttatcatcgcagatcctgagcttgtatggtgcactctcagtacaatctgctctgctgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgcccagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctgggtaccagctgctagcaagcttgctagcggccgctcgacctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgtcgaggccggcaaggccggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaatttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgactctaga

SEQ ID NO: 3 - DNA, 11687 bp, artificial sequence
  description of artificial sequence synthetic construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatccccataagcttccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgttgtgtcgctgtttgtggtggagcgagcaggccggcggaccctgcatctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgactcgacggtaccgcgggcccgggatccccgggtaccggaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaatttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactcgactctagagccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcggtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 4 - DNA, 11200 bp, artificial sequence
  description of artificial sequence synthetic construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaggtcgacggtatcgataagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgtcgacgcttgttctttttgcagaagctcagaataaacgctcaactagcttgatatcgaattcctgcagcccagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtccccgcgttgcttcctctgctggccgggacatcaggtggcccccgctgaattggaatcgatcagaattctgcagatatccatcacactggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactcgactctagagccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcggtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 5 - DNA, 12802 bp, artificial sequence
  description of artificial sequence synthetic construct
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgaggtcgacggtatcgataagcttccatggagcccagcagcaagaagctgacgggtcgcctcatgctggccgtgggaggagcagtgcttggctccctgcagtttggctacaacactggagtcatcaatgccccccagaaggtgatcgaggagttctacaaccagacatgggtccaccgctatggggagagcatcctgcccaccacgctcaccacgctctggtccctctcagtggccatcttttctgttgggggcatgattggctccttctctgtgggccttttcgttaaccgctttggccggcggaattcaatgctgatgatgaacctgctggccttcgtgtccgccgtgctcatgggcttctcgaaactgggcaagtcctttgagatgctgatcctgggccgcttcatcatcggtgtgtactgcggcctgaccacaggcttcgtgcccatgtatgtgggtgaagtgtcacccacagcccttcgtggggccctgggcaccctgcaccagctgggcatcgtcgtcggcatcctcatcgcccaggtgttcggcctggactccatcatgggcaacaaggacctgtggcccctgctgctgagcatcatcttcatcccggccctgctgcagtgcatcgtgctgcccttctgccccgagagtccccgcttcctgctcatcaaccgcaacgaggagaaccgggccaagagtgtgctaaagaagctgcgcgggacagctgacgtgacccatgacctgcaggagatgaaggaagagagtcggcagatgatgcgggagaagaaggtcaccatcctggagctgttccgctcccccgcctaccgccagcccatcctcatcgctgtggtgctgcagctgtcccagcagctgtctggcatcaacgctgtcttctattactccacgagcatcttcgagaaggcgggggtgcagcagcctgtgtatgccaccattggctccggtatcgtcaacacggccttcactgtcgtgtcgctgtttgtggtggagcgagcaggccggcggaccctgcacctcataggcctcgctggcatggcgggttgtgccatactcatgaccatcgcgctagcactgctggagcagctaccctggatgtcctatctgagcatcgtggccatctttggctttgtggccttctttgaagtgggtcctggccccatcccatggttcatcgtggctgaactcttcagccagggtccacgtccagctgccattgccgttgcaggcttctccaactggacctcaaatttcattgtgggcatgtgcttccagtatgtggagcaactgtgtggtccctacgtcttcatcatcttcactgtgctcctggttctgttcttcatcttcacctacttcaaagttcctgagactaaaggccggaccttcgatgagatcgcttccggcttccggcaggggggagccagccaaagtgacaagacacccgaggagctgttccatcccctgggggctgattcccaagtgtgactcgagatggggatccgtaatacgactcactatagggagatctctgcagccattcatctgatggccatcagatgaatggctgcagtctagagattctatagtgtcacctaaatgcaagcttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattgcggccgtcgacgcttgttctttttgcagaagctcagaataaacgctcaactagcttgatatcgaattcctgcagcccagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtccccgcgttgcttcctctgctggccgggacatcaggtggcccccgctgaattggaatcgatcagaattctgcagatatccatcacactggcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactcgactctagagccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcggtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 6 - DNA, 21 bp, artificial sequence
  description of artificial sequence synthetic construct
ataagcttccatggagcccag

SEQ ID NO: 7 - DNA, 24 bp, artificial sequence
  description of artificial sequence synthetic construct
atctcgagtcacacttgggaatca

SEQ ID NO: 8 - DNA, 25 bp, artificial sequence
  description of artificial sequence synthetic construct
ataagcttccatggacagtagcacc

SEQ ID NO: 9 - DNA, 26 bp, artificial sequence
  description of artificial sequence synthetic construct
atctcgagtcaggcaaaatatgcatg

SEQ ID NO: 10 - DNA, 19 bp, artificial sequence
  description of artificial sequence synthetic construct
gagcaggagaccaaacgac

SEQ ID NO: 11 - DNA, 19 bp, artificial sequence
  description of artificial sequence synthetic construct
gttgggtaggaagagatgg

SEQ ID NO: 12 - DNA, 4927 bp, artificial sequence
  description of artificial sequence synthetic construct
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatttaggtgacactatagaatctctagactgcagccattcatctgatggccatcagatgaatggctgcagagatctccctatagtgagtcgtattacggatccccatctcgagtcacacttgggaatcagcccccaggggatggaacagctcctcgggtgtcttgtcactttggctggctcccccctgccggaagccggaagcgatctcatcgaaggtccggcctttagtctcaggaactttgaagtaggtgaagatgaagaacagaaccaggagcacagtgaagatgatgaagacgtagggaccacacagttgctccacatactggaagcacatgcccacaatgaaatttgaggtccagttggagaagcctgcaacggcaatggcagctggacgtggaccctggctgaagagttcagccacgatgaaccatgggatggggccaggacccacttcaaagaaggccacaaagccaaagatggccacgatgctcagataggacatccagggtagctgctccagcagtgctagcgcgatggtcatgagtatggcacaacccgccatgccagcgaggcctatgaggtgcagggtccgccggcctgctcgctccaccacaaacagcgacacgacagtgaaggccgtgttgacgataccggagccaatggtggcatacacaggctgctgcacccccgccttctcgaagatgctcgtggagtaatagaagacagcgttgatgccagacagctgctgggacagctgcagcaccacagcgatgaggatgggctggcggtaggcgggggagcggaacagctccaggatggtgaccttcttctcccgcatcatctgccgactctcttccttcatctcctgcaggtcatgggtcacgtcagctgtcccgcgcagcttctttagcacactcttggcccggttctcctcgttgcggttgatgagcaggaagcggggactctcggggcagaagggcagcacgatgcactgcagcagggccgggatgaagatgatgctcagcagcaggggccacaggtccttgttgcccatgatggagtccaggccgaacacctgggcgatgaggatgccgacgacgatgcccagctggtgcagggtgcccagggccccacgaagggctgtgggtgacacttcacccacatacatgggcacgaagcctgtggtcaggccgcagtacacaccgatgatgaagcggcccaggatcagcatctcaaaggacttgcccagtttcgagaagcccatgagcacggcggacacgaaggccagcaggttcatcatcagcattgaattccgccggccaaagcggttaacgaaaaggcccacagagaaggagccaatcatgcccccaacagaaaagatggccactgagagggaccagagcgtggtgagcgtggtgggcaggatgctctccccatagcggtggacccatgtctggttgtagaactcctcgatcaccttctggggggcattgatgactccagtgttgtagccaaactgcagggagccaagcactgctcctcccacggccagcatgaggcgacccgtcagcttcttgctgctgggctccatggaagcttatgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggcgaatttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaattccaactgagcgccggtcgctaccattaccaacttgtctggtgtcaaaaataataggcctactagtcggccgtacgggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcggccttaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 13 - DNA, 7000 bp, artificial sequence
  description of artificial sequence synthetic construct
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttcctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaacccgtggtccccgcgttgcttcctctgctggccgggacatcaggtggcccccgctgaattggaatcgatcagaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc